(19) United States
(12) Patent Application Publication
(10) Pub. No.: US 2026/0256567 A1
(43) Pub. Date: Sep. 3, 2026

(54) IMPLANTABLE PROSTHESIS FOR MENDING DEFECTS OF SOFT TISSUE

(71) Applicant: Davol Inc., Warwick, RI (US)

(72) Inventors: Ricky N. Gomes, North Providence, RI (US); Pisit Khuon, Warwick, RI (US); Talia J. D'Ambruoso, San Jose, CA (US)

(73) Assignee: Davol Inc., Warwick, RI (US)

(21) Appl. No.: 18/878,537

(22) PCT Filed: May 18, 2023
(86) PCT No.: PCT/US2023/022690

§ 371 Date: Dec. 23, 2024

Related U.S. Application Data
(60) Provisional application No. 63/357,500, filed on Jun. 30, 2022.
(60) Provisional application No. 63/357,491, filed on Jun. 30, 2022.

(51) Int. Cl. A61F 2/00

(52) U.S. Cl. CPC A61F 2/0063; A61F 2/0077; A61F 2220/0008

(57) ABSTRACT

An implantable prosthesis for covering a defect in the peritoneum. The prosthesis may include a body (22) of implantable, biologically compatible material with first (24) and second surfaces (26). The first surface is tissue infiltratable and the second surface is adhesion resistant. The body includes a grip region adjacent its outer periphery with tissue grips (28) protruding in an outward direction away from the second surface. The prosthesis may be positioned between the peritoneum and a separate hernia repair fabric located in the preperitoneal pocket for repairing a tissue or muscle wall defect, such as a ventral hernia.

### CROSS-REFERENCE TO RELATED APPLICATIONS

This application claims the benefits of U.S. application No. 63/357,500, filed Jun. 30, 2022 and U.S. application No. 63/357,491, filed Jun. 30, 2022, both of which are hereby incorporated by reference in its entirety.

### FIELD

The present invention relates to an implantable prosthesis, and more particularly to a prosthesis for mending defects and weaknesses of soft tissue and muscle walls.

### BACKGROUND

A defect in a muscle or tissue wall, such as a hernia, is commonly repaired with an implantable prosthesis that is configured to cover and/or fill the defect.

For some procedures, an implantable repair fabric, such as a mesh fabric, is sutured, stapled, tacked, or otherwise provisionally anchored in place over, under or within the defect. Tissue integration with the fabric, such as tissue ingrowth into and/or along the mesh fabric, eventually completes the repair.

Various repair fabrics are known and used for repairing soft tissue and muscle wall defects. Examples of implantable fabrics that have been successfully used in soft tissue and muscle wall repair include BARD Soft Mesh, BARD Mesh and VISILEX, available from Davol, a division of Becton Dickinson. Such fabrics are fabricated from polypropylene monofilaments that are knitted into a mesh having pores or interstices that promote tissue ingrowth and integration with the fabric.

Ventral hernias may be repaired using a technique called a robotic transabdominal preperitoneal repair (rTAPP). This repair involves a surgeon creating a preperitoneal pocket within a patient for placement of a repair mesh. The mesh often does not include any anti-adhesion barrier or coating because it will be completely covered by the patient's peritoneum and not exposed to internal organs within the abdominal cavity. When forming the preperitoneal pocket, a potential drawback may be the creation of one or more small holes in the peritoneum during dissection by the surgeon. Such holes are often closed by the surgeon using a suture.

It is an object of the invention to provide a prosthesis and method for covering and/or mending defects in soft tissue and muscle walls, including the peritoneum.

### SUMMARY

The present disclosure relates to an implantable prosthesis and method for covering and/or mending an anatomical defect, such as a tissue or muscle wall defect, including a defect in the peritoneum.

According to one aspect, an implantable prosthesis comprises a body of implantable, biologically compatible material. The body includes a first surface and a second surface opposite the first surface. The first surface is tissue infiltratable and the second surface is adhesion resistant. The body includes an outer periphery and a grip region adjacent the outer periphery. A plurality of tissue grips are located within the grip region of the body and protrude in an outward direction away from the second surface.

According to another aspect, an implantable prosthesis comprises a body of implantable, biologically compatible material. The body includes a first surface and a second surface opposite the first surface. The first surface is tissue infiltratable and the second surface is resistant to tissue infiltration. The body has a maximum width of 5 cm or less.

According to another aspect, a method is provided for covering a defect in the peritoneum. The method comprises acts of: (a) forming a preperitoneal pocket between the peritoneum and a layer of tissue or muscle; and (b) placing at least one first implantable prosthesis within the preperitoneal pocket. The first implantable prosthesis includes a body of biologically compatible material with the body including a first surface and a second surface. The first surface is tissue infiltratable and the second surface is adhesion resistant. A plurality of tissue grips protrude in an outward direction away from the second surface. The method further comprises an act of (c) positioning the at least one first implantable prosthesis over at least one defect in the peritoneum with the first surface facing away from the peritoneum, the second surface facing the peritoneum, and the plurality of tissue grips engaging a region of the peritoneum adjacent the at least one peritoneum defect to maintain the first implantable prosthesis in position relative to the peritoneum defect.

According to another aspect, a method is provided for repairing a hernia defect. The method comprises acts of: (a) forming a preperitoneal pocket between the peritoneum and a layer of tissue or muscle having a hernia defect; and (b) placing at least one first implantable prosthesis within the preperitoneal pocket. The first implantable prosthesis includes a body of biologically compatible material including a first surface and a second surface with the second surface being adhesion resistant. The method further comprises an act of: (c) placing a second implantable prosthesis within the preperitoneal pocket. The second implantable prosthesis includes a body of biologically compatible material which is tissue infiltratable for repairing the hernia defect. The method further comprises acts of: (d) positioning the at least one first implantable prosthesis over at least one defect in the peritoneum with the first surface facing away from the peritoneum and the second surface facing the peritoneum; and (e) positioning the second implantable prosthesis over the hernia defect in the layer of tissue or muscle.

The foregoing is a non-limiting summary of the disclosure, which is defined by the attached claims. Other aspects, embodiments and/or features will become apparent from the following description.

Various embodiments of the present disclosure may provide certain advantages and may overcome certain drawbacks of prior prostheses. Embodiments of the disclosure may not share the same advantages, and those that do may not share them under all circumstances.

### BRIEF DESCRIPTION OF DRAWINGS

Aspects of the disclosure are described below, by way of example, with reference to the accompanying drawings in which like numerals reference like elements, and wherein:

FIG. 1 is a front view of an implantable prosthesis according to one embodiment;

FIG. 2 is a rear view of the implantable prosthesis of FIG. 1;

FIG. 3 is an enlarged sectional view taken along section line 3-3 of FIG. 2 illustrating tissue grips located along a peripheral region of the prosthesis;

FIG. 4 is a schematic view of a human body illustrating the region of a preperitoneal pocket formed about a ventral hernia;

FIG. 5 is an illustration of a peritoneum with multiple holes created during formation of a peritoneal pocket;

FIG. 6 is an illustration of the peritoneum of FIG. 6 with the implantable prosthesis of FIGS. 1-3 positioned over the holes; and

FIG. 7 is an exploded, schematic view of a ventral hernia repair employing the implantable prosthesis of FIGS. 1-3.

### DETAILED DESCRIPTION

It should be understood that aspects of the disclosure are described herein with reference to the figures, which show illustrative embodiments in accordance with aspects of the disclosure. The illustrative embodiments described herein are not necessarily intended to show all aspects of the disclosure, but are used to describe a few illustrative embodiments. Thus, aspects of the disclosure are not intended to be construed narrowly in view of the illustrative embodiments. It should be appreciated, then, that the various concepts and embodiments discussed herein may be implemented in any of numerous ways, as the disclosed concepts and embodiments are not limited to any particular manner of implementation. In addition, it should be understood that aspects of the disclosure may be used alone or in any suitable combination with other aspects of the disclosure.

The disclosure is directed to an implantable prosthesis for covering and/or mending an anatomical defect, and is particularly suitable for covering and/or mending defects in, and weaknesses of, soft tissue and muscle walls or other anatomical regions. The phrase “mending a defect” includes acts of repairing, augmenting, and/or reconstructing a defect and/or a potential defect. For ease of understanding, and without limiting the scope of the invention, the prosthesis is described below particularly employed in connection with a defect in the peritoneum which may occur during the formation of a preperitoneal pocket in conjunction with a ventral hernia repair including, but not limited to, one or more of an umbilical hernia, an incisional hernia and/or other weakness or rupture of the abdomen. It should be understood, however, that the prosthesis is not so limited and may be employed in other anatomical procedures, as should be apparent to one of skill in the art. For example, and without limitation, the prosthesis may be employed for inguinal hernias, chest or abdominal wall reconstruction, or large defects, such as those that may occur in obese patients. The prosthesis may include one or more features, each independently or in combination, contributing to such attributes.

The disclosure is more particularly directed to a prosthesis which includes a body of biologically compatible material, such as a repair fabric, which is configured to cover or extend across the defect opening or weakness when the body is placed against the defect. The prosthesis may be in the form of a patch, although the prosthesis may employ other configurations as should be apparent to one of skill in the art. The patch may have a planar or non-planar configuration suitable for a particular procedure employed for mending a defect.

The prosthesis may be configured with a self-grip arrangement having features that help maintain the position of the prosthesis relative to the defect. The self-grip arrangement may reduce, if not eliminate, separation, sliding, twisting, folding and/or other movement, as may be desired, between the prosthesis and adjacent tissue. Such an arrangement may also reduce, if not eliminate, the need for a surgeon to suture, staple, tack, or otherwise provisionally anchor the prosthesis in place pending tissue integration.

The prosthesis may include a plurality of grips positioned on the body. More particularly, the grips may be located on a surface of the body that is configured to be placed against adjacent muscle and/or tissue.

For some applications, the grips may include a microstructure arrangement capable of gripping low coefficient of friction materials without causing damage to the material, including tissue and/or muscle. In some embodiments, the microstructure arrangement may be configured to grip tissue using fluid mechanics. For example, the microstructure texture of the grip may allow fluid to penetrate into the microstructure providing a high solid-fluid adhesion and high contact angle hysteresis.

For some applications, the grips may be configured to penetrate and grip the tissue when the prosthesis is placed and/or pressed against it. In some embodiments, the grips may be configured to protrude a defined distance from the surface of the body portion to grasp onto and/or penetrate a depth of tissue sufficient to provide the desired amount of grip. For example, the grips may be configured as barbs, hooks or other suitable structures as should be apparent to one of skill.

The grips may be arranged on the body in any suitable configuration to provide a desired amount of grip as should be apparent to one of skill in the art. For example, and without limitation, the grips may be distributed across the body in a uniform, non-uniform or random array, and/or any suitable combination of arrays. The grips may be distributed across the entire body or located at one or more select regions of the body. For example, and without limitation, the grips may be located at one or more select regions adjacent one or more portions of the outer periphery of the body, and/or one or more select regions located within the inner region of the body inwardly away from the outer periphery. Each select region may include one or more grips arranged in any suitable pattern within the region. One or more of the select regions may employ the same or different arrangements of grips relative to one or more other select regions of grips.

According to one aspect, the grips may be located within a peripheral grip region of the body which is located adjacent to and extends inwardly from its outer periphery. An inner region of the body may be free of grips. In this manner, the grip region may be configured and arranged to provide a sufficient amount of grip with minimal coverage of the prosthetic body. According to one aspect, the area of the grip region may be proportional to the size of the body.

According to one aspect, the prosthetic body may be configured with a circular shape and the grip region may be configured with an annular shape located adjacent the outer periphery of the body. The annular shape of the grip region may be configured to extend about and not overlie the defect when the body is positioned over the defect. According to one aspect, the grip region may be configured to surround the defect and have an inner periphery which is larger than the size of the defect. In this manner, the grip region may be configured to avoid direct engagement with any portion of the defect. However, it is to be understood that the body and/or the grip region may employ any configuration and/or size suitable for a particular repair as should be apparent to one of skill in the art.

According to one aspect, the grips may be fabricated independent of and mounted to the body of the prosthesis. In this manner, the grips may be formed from a material that is different from the body. For example, and without limitation, the grips and the body may both be formed of a bioresorbable material, with the grips having the same or a different degradation time relative to the body. For example, and without limitation, the grips may be formed of a material which is resorbed faster than the body of the prosthesis. For some applications, the grips may be formed of a bioresorbable material, while the body may be formed of a non-resorbable material. Such arrangements may provide the prosthesis with temporary grip properties during the period of tissue integration, while reducing the amount of foreign material that remains present in a patient's body and maintaining longer-term strength of the prosthesis.

Independent fabrication of the grips may also provide flexibility for configuring the prosthesis. For example, and without limitation, the prosthesis may include grips having the same or different grip configurations and/or arrangements depending on a particular application of the prosthesis. For example, and without limitation, the prosthesis may include grips having the same shape, but mounted in different orientations relative to each other on the body. The prosthesis may include grips with one or more different shapes in one or more regions of the body. In this manner, the prosthesis may be provided with various grip characteristics based on the particular orientations and/or shapes of the grips individually and as a whole.

The grips may be directly or indirectly mounted to the body of the prosthesis. According to one aspect, the prosthesis may include a grip segment which includes multiple grips provided on a common base. For example, and without limitation, each grip may include a grip body extending from a base of material including, but not limited to, a film material. The base may be mounted to the body with the grip body protruding away from a surface of the body and configured to engage and grip tissue. The grip segment may be molded with the grips and the base forming a single, unitary structure.

In one aspect, the grips may be molded or imprinted directly on the body. For example, and without limitation, the grips may be insert molded to a prefabricated mesh fabric placed within a mold that receives grip material and forms the desired shape of the grip directly onto the body. Alternatively, the grips may be imprinted directly onto the material of the repair fabric or imprinted onto a layer of material which is attached to the repair fabric. For example, and without limitation, the repair fabric may include a coating or layer of material, such as an adhesion resistant material, onto which the grips may be imprinted. This arrangement would reduce the need for another component and processing step.

In one aspect, the grips may be mechanically mounted to the body. For example, and without limitation, the grips may be mounted using a mechanical component or arrangement that is attached to the body. For example, and without limitation, the body may include a layer of repair fabric and the grips may be independently formed and secured to the fabric layer. As indicated above, the grips may be provided on a grip segment including a base material which is subsequently secured to the layer of repair fabric. Such indirect mounting arrangements may reduce the likelihood of potential degradation of the strength, tissue infiltration characteristics and/or adhesion resistant characteristics of the layer of repair fabric.

In one embodiment, the grips may be formed on a layer of film material which is attached to the layer of repair fabric. The grips may be integrated as part of the film layer by molding or imprinting the grips on the film.

The prosthesis may be used for covering and/or mending soft tissue and muscle wall defects using various surgical techniques, including open, laparoscopic, hybrid (e.g., Kugel procedure), and robotic techniques. During open procedures, the prosthesis may be placed through a relatively large incision made in the abdominal wall and layers of tissue and then the defect is filled or covered with the repair fabric. During laparoscopic and hybrid procedures, the prosthesis may be collapsed, such as by rolling or folding, into a reduced configuration for entry into a subject, either directly through a comparatively smaller incision or through a slender laparoscopic cannula that is placed through the incision.

The prosthesis may have particular application with robotic procedures in which placement of the prosthesis is achieved using surgical robotic tools which may involve passage of the prosthesis through a relatively small cannula (e.g., 8 mm) as compared to a cannula (e.g., 10-12 mm) typically employed for more conventional laparoscopic techniques. The prosthesis may be particularly suitable for use in a rTAPP procedure in which one or more holes may be formed in the peritoneum during creation of the preperitoneal pocket. In this regard, the prosthesis may be employed to cover holes formed in the peritoneum to isolate viscera located on one side of the peritoneum from either exposed tissue repair fabric, such as hernia repair mesh, or tissue located on the opposite side of the peritoneum. For example, and without limitation, the prosthesis may be employed to prevent direct contact between viscera and exposed mesh fabric used to repair a hernia or other tissue defect within the preperitoneal pocket.

FIGS. 1-3 illustrate one embodiment of a prosthesis for covering and/or mending tissue and muscle wall defects including, without limitation, a defect in the peritoneum which may be formed during a preperitoneal hernia repair procedure. The prosthesis 20 may include a body 22 of implantable, biologically compatible material. In one embodiment, the body 22 may include a repair fabric which is relatively flexible, thin and light weight and meets the performance and physical characteristics for covering and/or mending soft tissue and muscle wall defects.

The body 22 may be configured with a size and/or shape suitable to cover or extend across the opening or weakness in tissue and/or muscle when the body is placed against the tissue and/or muscle wall with the defect. The body may include a first surface 24 and a second surface 26 opposite the first surface. In one embodiment, the first surface 24 may be tissue infiltratable and the second surface 26 may be adhesion resistant. In this manner, and without limitation, the prosthesis may be positioned at a repair site with the first surface facing soft tissue and/or muscle for receiving tissue ingrowth and with the second surface facing the viscera, or other organs, tissue and/or muscle, to avoid tissue adhesions to the body.

The prosthesis may also include a plurality of grips 28 on a surface of the body to provide a self-grip arrangement for maintaining the position of the prosthesis relative to the defect. In one embodiment, the grips 28 may be provided on the second surface 26 of the body for engaging adjacent tissue, such as the peritoneum. In this manner, the grips may be provided on the adhesion resistant surface of the body to grip the peritoneum with the adhesion resistant surface facing the viscera on the opposite side of the peritoneum when the prosthesis is implanted within the preperitoneal pocket to cover one or more holes in the peritoneum. However, it is to be appreciated that the grips may be provided on the tissue infiltratable surface of the prosthesis, if desired, for other applications.

The grips 28 may be configured to grip the tissue when the prosthesis is placed and/or pressed against it. In this manner, the grips may be configured to protrude from the surface of the body to engage and/or interact with tissue sufficiently to provide the desired amount of grip. In one embodiment, the grips 28 may include a microstructure arrangement configured to grip low coefficient of friction materials without causing damage to the material, including tissue and/or muscle. The microstructure arrangement may be configured to grip tissue using fluid mechanics. For example, and without being bound by theory, the microstructure texture of the grip may allow fluid to penetrate into the microstructure to provide a high solid-fluid adhesion and high contact angle hysteresis.

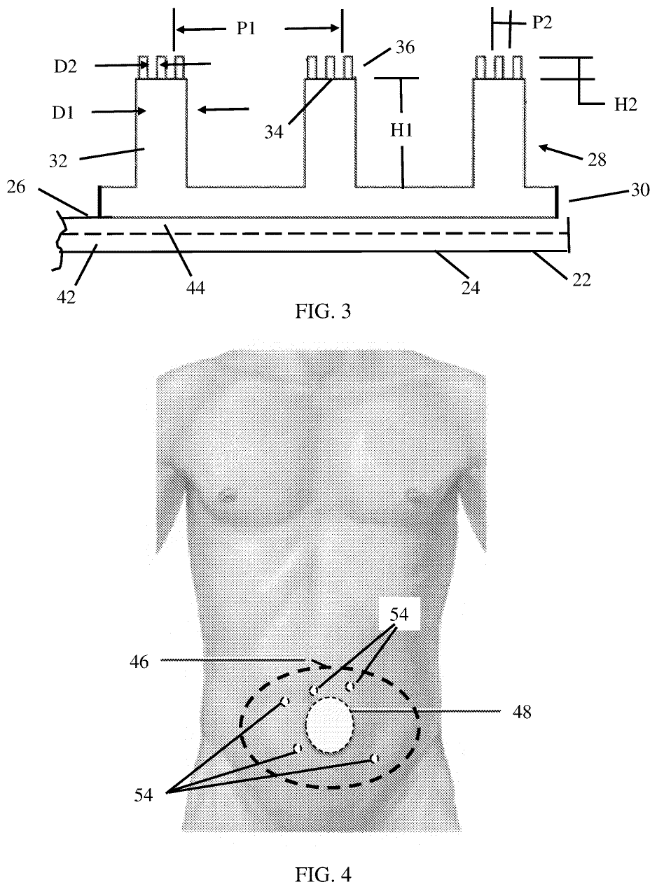

In one embodiment illustrated in FIG. 3, the microstructure arrangement may include multiple grips 28 protruding outwardly from a base 30 to form a grip segment. Each grip 28 may include a first or primary pillar 32 protruding from the base 30 with a distal end 34 of the primary pillar spaced from the base. Each grip 28 may also include a plurality of second or secondary pillars 36 protruding from a surface of the primary pillar 32. As illustrated, the secondary pillars 34 may extend from the distal end of the primary pillar. The grip segment including the grips and the base may be fabricated from a film material.

In one illustrative embodiment as shown in FIG. 3, each primary pillar 32 may have a cylindrical shape. In some embodiments, the primary pillars may have a diameter D1 of approximately 0.09 inches to approximately 0.11 inches and may be spaced apart at a pitch P1 of approximately 0.18 inches to approximately 0.22 inches. The primary pillars may have a height H1 of approximately 0.045 inches to approximately 0.055 inches. The primary pillars may have a shaft angle of approximately 3° to approximately 7°.

In one illustrative embodiment, the secondary pillars 36 may also have a cylindrical shape and extend from a surface of the primary pillar in a substantially perpendicular direction. In some embodiments, the secondary pillars may have a diameter D2 of approximately 0.009 inches to approximately 0.011 inches and may be spaced apart at a pitch P2 of approximately 0.018 inches to approximately 0.022 inches. The secondary pillars may have a height H2 of approximately 0.018 inches to approximately 0.022 inches.

It is to be appreciated that other cross-sectional shapes (e.g., square, elliptical, orthogonal, etc.), dimensions, and patterns for the primary and/or secondary pillars are also contemplated, as the disclosure is not so limited. It is also to be appreciated that the secondary pillars may extend from any suitable surface of the primary pillar and/or at an angle which is not perpendicular to the surface. Although the segment shown in FIG. 3 includes three grips, it is to be understood that this is for illustrative purposes only, and the grip segment may include any number and density of grips suitable for a particular application.

According to one aspect, the grips 28 may be located at one or more grip regions of the body. Such an arrangement may be suitable for placing grips in selected regions of the body to accommodate a particular anatomical region. For example, it may be desirable to avoid providing grips on regions of the body portion that may potentially contact the defect, vessels, nerves or other portions of the anatomy at the defect site as should be apparent to one of skill in the art.

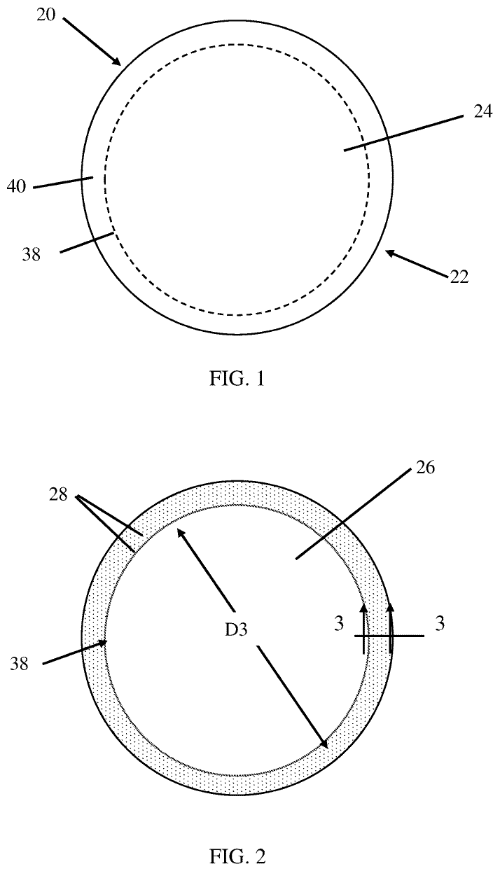

As illustrated in FIGS. 1-2, the prothesis may include a circular body with the grips being located within an annular grip region 38 adjacent the outer periphery 40 of the body 22. In one embodiment, the grip region may extend continuously about the entire periphery of the body. However, it is to be understood that the grips may be located in two or more regions of the body. In this regard, multiple grip segments may be located at desired location of the body. Moreover, it is to be appreciated that the prosthesis may employ a body and/or grip region having any shape suitable for a particular application.

The body 22 may have any size suitable for the particular repair and sizes of holes in the peritoneum. In one embodiment, particularly suited for covering holes in the peritoneum, the body may have a width ranging from 1 cm to 5 cm. In some embodiments, the body may have a maximum width of 5 cm or less, 4 cm or less, 3 cm or less, 2 cm or less, 1 cm or more, 2 cm or more, 3 cm or more and 4 cm or more. For a circular body, it is to be understood that the maximum width corresponds to the outer diameter.

The body may be configured to include a grip region 38 sufficient to provide a desirable amount of tissue grip to maintain the prosthesis in position relative to defect(s). In one embodiment, the grip region may be configured to occupy approximately 10% to approximately 20% of the total area of the first surface 24 of the body. For a circular body having a diameter ranging from 1 cm to 5 cm, the grip region may be configured to occupy from approximately 0.1 cm 2 to approximately 3.92 cm 2 of the body area.

As indicated above, the grips 28 may be arranged in an annular grip region 38 adjacent the outer periphery 40 of the body. The inner diameter D3 of the annular grip region may about 0.90 cm to 0.95 cm for a 1 cm diameter body, about 1.79 cm to 1.90 cm for a 2 cm diameter body, about 2.68 cm to 2.85 cm for a 3 cm diameter body, about 3.58 cm to 3.80 cm for a 4 cm diameter body, and about 4.47 cm to 4.75 cm for a 5 cm diameter body. It is to be appreciated that the grip region may be configured to have any suitable shape and/or size relative to the body as should be apparent to one of skill in the art.

The body 22 may include a mesh fabric and may employ a knit construction that provides openings or pores to allow tissue infiltration to incorporate the prosthesis. The repair fabric may also have sufficient flexibility to promote an easy reduction in size for entry into the subject. In this manner, the flexible fabric may be collapsed into a slender configuration, such as a roll, which can be supported in, and advanced through, a narrow laparoscopic cannula for use in laparoscopic or robotic procedures.

In one embodiment, the body 22 of the prosthesis may be formed from a layer of PHASIX ST mesh (available from Davol, Inc.) which is a resorbable repair fabric having a first surface 24 that supports functional healing via tissue ingrowth and a second surface 26 with a hydrogel barrier that is adhesion resistant to minimize tissue attachment. When implanted, the mesh promotes rapid tissue or muscle ingrowth into and around the mesh structure while minimizing potential attachment to adjacent viscera. The prosthesis eventually becomes resorbed at which time the prosthesis is no longer necessary to separate the viscera on one side of the peritoneum from the repair mesh and/or tissue on the opposite side of the peritoneum. For example, and without limitation, the prosthesis may not be resorbed until such time that the hernia repair mesh has become integrated with the tissue and/or has become resorbed itself so as to no longer be susceptible to adhesions with the viscera.

As illustrated in FIG. 3, a separate grip segment may be mounted on the second surface 26 of the body with the base 30 of the grip segment being attached to the body. However, it is to be appreciated that the grips may be integrated with the body, for example, by molding or imprinting the grip on the body, as should be understood by one of skill in the art.

For some applications, the prosthesis 20 may be formed from two or more layers of biocompatible material. For example, and without limitation, the body 22 of the prosthesis may include a first or ingrowth layer 42 of knitted mesh fabric and a second or barrier layer 44 of adhesion resistant material overlying a surface of the mesh fabric. The barrier layer may be separate from and attached to the fabric layer using any suitable fastening technique as should be apparent to one of skill in the art. For example, and without limitation, the mesh fabric and the barrier layer may be joined together by suturing, ultrasonic welding, lamination and/or overmolding.

As illustrated in FIG. 3, a grip segment may overlie and be mounted on the barrier layer. However, for some applications, the barrier layer may be smaller than the ingrowth layer with the grip segment mounted on the growth layer.

Examples of resorbable surgical materials which may be utilized for the mesh fabric layer and are suitable for tissue or muscle reinforcement and defect correction include, but are not limited to, PHASIX Mesh (available from Davol, Inc.), polyglactin (VICRYL—available from Ethicon, Inc.) and polyglycolic acid (DEXON—available from US Surgical, Inc.). Collagen materials such as COOK SURGISIS, available from Cook Biomedical, Inc. may also be used. Non-resorbable materials, including BARD Mesh (available from Davol, Inc.), BARD Soft Mesh (available from Davol, Inc.), SOFT TISSUE PATCH (microporous ePTFE—available from W.L. Gore & Associates, Inc.); SURGIPRO (available from US Surgical, Inc.); TRELEX (available from Meadox Medical); PROLENE and MERSILENE (available from Ethicon, Inc.); and other mesh materials (e.g., available from Atrium Medical Corporation), may be suitable for applications involving permanent correction of tissue or muscle defects. It also is contemplated that the mesh fabric may be formed from multifilament yarns and that any suitable method, such as knitting, weaving, braiding, molding and the like, may be employed to form the mesh material.

For some embodiments employing a separate barrier layer which may be attached to a mesh fabric layer, the barrier may be formed from a sheet of expanded polytetrafluoroethylene (ePTFE), such as GORETEX available from W.L. Gore & Associates, Inc., having a pore size (submicronal) that discourages tissue ingrowth and adhesion. A representative and non-limiting sampling of other suitable barrier materials includes silicone elastomer, such as SILASTIC Rx Medical Grade Sheeting (Platinum Cured) distributed by Dow Corning Corporation, TEFLON mesh, and microporous polypropylene sheeting (CELGARD). Autogenous, heterogenous and xenogeneic tissue also are contemplated including, for example, pericardium and small intestine submucosa.

Absorbable materials, such as oxidized, regenerated cellulose (Intercede (TC7)) may be employed for some applications. It is to be appreciated that any suitable adhesion resistant materials may be used as should be apparent to one of skill in the art.

In one embodiment, the grips 28 and the base 30 may be formed of a resorbable material. In this manner, the grips provide a desired level of tissue grip for placement and positioning of the prosthesis. The grips and the base eventually become resorbed at which time the prosthesis has become sufficiently integrated with the tissue. In some embodiments, the grips 28 and the base 30 may be made from PURASORB® PLDL 7038, a GMP grade copolymer of L-lactide and DL-lactide in a 70/30 molar ratio and with an inherent viscosity midpoint of 3.8 dl/g. Examples of other resorbable materials which may be utilized for the grips include, without limitation, PLG (L-lactide/glycolide) or PLA (polylactic acid) polymers. For some applications, it may be desirable to utilize non-resorbable grips. An example of non-rebsorbable material which may be utilized for the grips includes, but without limitation, polypropylene. It is to be appreciated that other suitable biocompatible materials, resorbable or non-resorbable, may be used for the grips and/or grip segment as should be apparent to one of skill in the art.

In one embodiment, the grips may be fabricated using an injection molding process. However, any suitable manufacturing process may be used to fabricate the grips as should be apparent to one of skill in the art. For example, and without limitation, the grips may be fabricated using embossing or 3D printing processes.

As indicated above, the prosthesis may be suitable for use during a hernia repair procedure, including a ventral hernia repair, which involves the creation of a preperitoneal pocket. One particular procedure may involve a robotic transabdominal preperitoneal repair (rTAPP). This repair involves a surgeon creating a preperitoneal pocket within a patient for placement of a primary repair mesh. The mesh often does not include any anti-adhesion barrier or coating because it will be completely covered by the patient's peritoneum and not exposed to internal organs or viscera within the abdominal cavity. However, forming the preperitoneal pocket may result in the creation of one or more small holes in the peritoneum during dissection by the surgeon.

The prosthesis 20 may be employed to cover holes formed in the peritoneum and prevent contact between the hernia repair mesh and underlying viscera through the holes in the peritoneum. In this manner, the prosthesis provides a barrier between the viscera and the hernia repair mesh should one or more holes be inadvertently created in the peritoneum during the repair procedure.

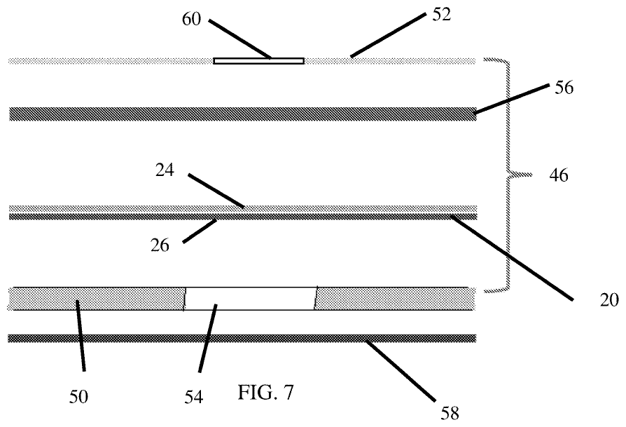

In one embodiment illustrated in FIGS. 4-7, a repair procedure involves the repair of an umbilical hernia and, more particularly a hernia in the posterior rectus sheet. As illustrated in FIG. 4, a preperitoneal pocket 46 (shown in phantom) may be formed in the region of the umbilical hernia 48 following conventional surgical techniques as should be understood by one of ordinary skill in the art. This involves performing a dissection to create a tissue plane between the peritoneum 50 and the posterior rectus sheet 52, as shown in FIG. 7.

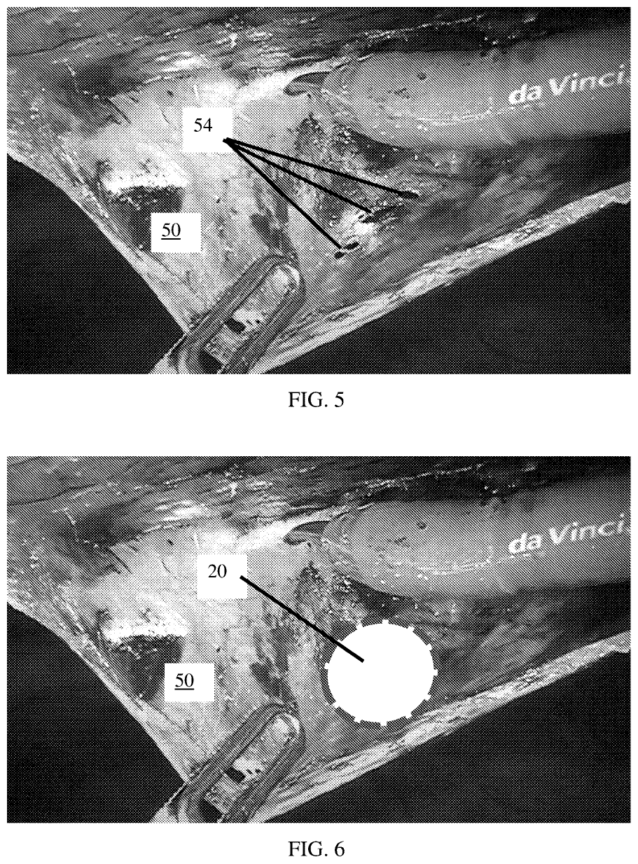

As shown in FIGS. 4 and 5, formation of the pocket 46 may result in the creation of one or more holes 54 in the peritoneum 50 as it is dissected away from the posterior rectus sheet.

As illustrated in FIGS. 6 and 7, the hole(s) formed in the peritoneum may be covered with one or more of the prostheses 20 according to the disclosure. More particularly, a prosthesis 20 may be placed within the preperitoneal pocket 46 and positioned over the hole(s) 54 with the barrier surface 26 facing the peritoneum and the grips 28 placed in contact with the peritoneum. The tissue ingrowth surface 24 of the prosthesis faces away from the peritoneum 50 toward the overlying hernia repair fabric 56 and/or tissue.

As illustrated in FIG. 7, a hernia repair mesh 56 is placed in the preperitoneal pocket 46 and positioned over a hernia defect 60 in posterior rectus sheath 52. The hernia repair mesh 56 is tissue infiltratable to support ingrowth of tissue for repairing the hernia defect 60 in the posterior rectus sheath 52 as should be understood by one of ordinary skill in the art.

As illustrated, the prosthesis 20 is positioned between the hole 54 in the peritoneum and the hernia repair mesh to separate the hernia repair mesh and viscera 58 exposed by the hole in the peritoneum.

The particular sequence of placing and positioning one or more prostheses and the hernia repair mesh within the preperitoneal pocket is at the discretion of the surgeon. For example, and without limitation, one or more prostheses 20 may be placed within the preperitoneal pocket and positioned over any holes created in the peritoneum prior to, after, or concurrently with introducing the hernia repair mesh into the preperitoneal pocket and/or positioning the hernia repair mesh over the hernia defect.

For this particular repair, the prosthesis 20 is intended to function as a barrier between the mesh fabric 56 for repairing the hernia defect and the viscera 58 exposed by holes 54 in the peritoneum. In this manner, the prosthesis may be used as a type of wound covering or bandage to prevent adhesions between the hernia repair mesh and the viscera. However, it is to be appreciated that the prosthesis may be employed as a repair fabric for mending a defect in other applications as should be apparent to one of skill in the art.

For purposes of this patent application and any patent issuing thereon, the indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

The use of “including,” “comprising,” “having,” “containing,” “involving,” and/or variations thereof herein, is meant to encompass the items listed thereafter and equivalents thereof as well as additional items.

It should also be understood that, unless clearly indicated to the contrary, in any methods claimed herein that include more than one step or act, the order of the steps or acts of the method is not necessarily limited to the order in which the steps or acts of the method are recited.

The foregoing description of various embodiments are intended merely to be illustrative thereof and that other embodiments, modifications, and equivalents are within the scope of the invention recited in the claims appended hereto.

1. An implantable prosthesis comprising:
a body of implantable, biologically compatible material, the body including a first surface and a second surface opposite the first surface, the first surface being tissue infiltratable, the second surface being adhesion resistant; and
a plurality of tissue grips protruding in an outward direction away from the second surface.

2. The implantable prosthesis according to claim 1, wherein the body includes an outer periphery and a grip region adjacent the outer periphery, the plurality of tissue grips being confined within the grip region.

3. The implantable prosthesis according to claim 2, wherein the body has a circular shape and the grip region has an annular shape.

4. The implantable prosthesis according to claim 2, wherein the first surface has a total surface area, the grip region being approximately 10% to approximately 20% of the total surface area.

5. The implantable prosthesis according to claim 1, wherein the body has a maximum width of 5 cm or less.

6. The implantable prosthesis according to claim 1, wherein the body has a maximum width of 1 cm to 5 cm.

7. The implantable prosthesis according to claim 5, wherein the body has a circular shape with an outer diameter, the maximum width corresponding to the outer diameter.

8. The implantable prosthesis according to claim 1, wherein the plurality of tissue grips are independent of the body.

9. The implantable prosthesis according to claim 1, wherein the plurality of tissue grips are integrated with the body.

10. The implantable prosthesis according to claim 9, wherein the plurality of tissue grips are molded to the body.

11. The implantable prosthesis according to claim 1, wherein the plurality of tissue grips are microstructure grips.

12. The implantable prosthesis according to claim 11, wherein each of the plurality of tissue grips includes a first pillar and a plurality of second pillars extending from a surface of the first pillar.

13. The implantable prosthesis according to claim 1, wherein the plurality of tissue grips are resorbable.

14. The implantable prosthesis according to claim 1, wherein the body is resorbable.

15. A method of covering a defect in a peritoneum, the method comprising acts of:
(a) forming a preperitoneal pocket between the peritoneum and a layer of tissue or muscle;
(b) placing at least one first implantable prosthesis within the preperitoneal pocket, the first implantable prosthesis including a body of biologically compatible material, the body including a first surface and a second surface, the first surface being tissue infiltratable and the second surface being adhesion resistant, the implantable prosthesis further including a plurality of tissue grips protruding in an outward direction away from the second surface;
(c) positioning the at least one first implantable prosthesis over at least one defect in the peritoneum with the first surface facing away from the peritoneum and the second surface facing the peritoneum with the plurality of tissue grips engaging a region of the peritoneum adjacent the at least one defect to maintain the at least one first implantable prosthesis in position relative to the at least one defect.

16. The method according to claim 15, wherein act (c) includes placing the second surface against the peritoneum.

17. The method according to claim 15, wherein the peritoneum includes a plurality of defects, wherein act (b) includes placing a plurality of first implantable prostheses within the preperitoneal pocket, and wherein act (c) includes positioning each of the plurality of first implantable prostheses over each of the defects in the peritoneum.

18. The method according to claim 15, further comprising an act (d) of placing a second implantable prosthesis within the preperitoneal pocket, the second implantable prosthesis including a body of biologically compatible material which is tissue infiltratable.

19. The method according to claim 18, further comprising an act (e) of positioning the second implantable prosthesis at a hernia defect in the layer of tissue or muscle.

20. The method according to claim 18, wherein act (c) includes positioning one or more first implantable prostheses over one or more defects in the peritoneum and between the second implantable prosthesis and the peritoneum.

21. The method according to claim 15, wherein the layer of tissue or muscle is the posterior rectus sheath.

22. The method according to claim 15, wherein the method is a robotic transabdominal preperitoneal repair (rTAPP) procedure.

23. The method according to claim 15, wherein the body has an outer periphery and a grip region adjacent the outer periphery, the plurality of tissue grips being confined within the grip region

24. The method according to claim 23, wherein the body has a circular shape and the grip region has an annular shape.

25. The method according to claim 23, wherein the first surface has a total surface area, the grip region being approximately 10% to approximately 20% of the total surface area.

26. The method according to claim 15, wherein the plurality of tissue grips protrude in an outward direction relative to the second surface.

27. The method according to claim 15, wherein the body of the first implantable prosthesis has a maximum width of 5 cm or less.

28. The method according to claim 27, wherein the maximum width of the body is 4 cm or less.

29. The method according to claim 27, wherein the maximum width of the body is 3 cm or less.

30. The method according to claim 27, wherein the maximum width of the body is 2 cm or less.

31. The method according to claim 27, wherein the maximum width of the body is 1 cm or more.

32. The method according to claim 27, wherein the maximum width of the body ranges from 1 cm to 5 cm.

33. The method according to claim 27, wherein the body has a circular shape with an outer diameter, the maximum width corresponding to the outer diameter.

34. The method according to claim 15, wherein the plurality of grips are microstructure grips.

35. The method according to claim 34, wherein each of the plurality of tissue grips includes a first pillar and a plurality of second pillars extending from a surface of the first pillar.

36. The method according to claim 15, wherein the plurality of tissue grips are resorbable.

37. The method according to claim 15, wherein the body is resorbable.

38. An implantable prosthesis comprising:
a body of implantable, biologically compatible material, the body including a first surface and a second surface opposite the first surface, the first surface being tissue infiltratable, the second surface being resistant to tissue infiltration, the body having a maximum width of 5 cm or less.

39. The implantable prosthesis according to claim 38, wherein the body has a maximum width of 4 cm or less.

40. The implantable prosthesis according to claim 38, wherein the body has a maximum width of 3 cm or less.

41. The implantable prosthesis according to claim 38, wherein the body has a maximum width of 2 cm or less.

42. The implantable prosthesis according to claim 38, wherein the body has a maximum width of 1 cm to 5 cm.

43. The implantable prosthesis according to claim 38, wherein the body has a circular shape with an outer diameter, the maximum width corresponding to the outer diameter.

44. The implantable prosthesis according to claim 38, further comprising a plurality of tissue grips protruding in an outward direction away from the second surface of the body.

45. The implantable prosthesis according to claim 44, wherein the body includes an outer periphery and a grip region adjacent the outer periphery, the plurality of tissue grips being confined within the grip region.

46. The implantable prosthesis according to claim 45, wherein the body has a circular shape and the grip region has an annular shape.

47. The implantable prosthesis according to claim 45, wherein the first surface has a total surface area, the grip region being approximately 10% to approximately 20% of the total surface area.

48. The implantable prosthesis according to claim 44, wherein the plurality of tissue grips are microstructure grips.

49. The implantable prosthesis according to claim 48, wherein each of the plurality of tissue grips includes a first pillar and a plurality of second pillars extending from a surface of the first pillar.

50. The implantable prosthesis according to claim 38, wherein the plurality of tissue grips are resorbable.

51. The implantable prosthesis according to claim 38, wherein the body is resorbable.

52. A method of repairing a hernia defect, the method comprising acts of:
(a) forming a preperitoneal pocket between a peritoneum and a layer of tissue or muscle having a hernia defect;
(b) placing at least one first implantable prosthesis within the preperitoneal pocket, the first implantable prosthesis including a body of biologically compatible material, the body including a first surface and a second surface, the second surface being adhesion resistant;
(c) placing a second implantable prosthesis within the preperitoneal pocket, the second implantable prosthesis including a body of biologically compatible material which is tissue infiltratable for repairing the hernia defect;
(d) positioning the at least one first implantable prosthesis over at least one defect in the peritoneum with the first surface facing away from the peritoneum and the second surface facing the peritoneum; and
(e) positioning the second implantable prosthesis over the hernia defect in the layer of tissue or muscle.

53. The method according to claim 52, wherein the at least one first implantable prosthesis further includes a plurality of tissue grips protruding in an outward direction away from the second surface.

54. The method according to claim 53, wherein act (d) includes engaging the plurality of tissue grips with a region of the peritoneum adjacent the at least one defect in the peritoneum to maintain the at least one first implantable prosthesis in position relative to the defect in the peritoneum.

55. The method according to claim 53, wherein the plurality of grips are microstructure grips.

56. The method according to claim 53, wherein the body of the at least one first implantable prothesis has a circular shape with an outer periphery and includes an annular grip region adjacent the outer periphery, the plurality of grips being confined to the grip region.

57. The method according to claim 56, wherein the first surface of the body has a total surface area, the grip region being approximately 10% to approximately 20% of the total surface area.

58. The method according to claim 53, wherein the plurality of tissue grips protrude in an outward direction relative to the second surface.

59. The method according to claim 53, wherein the plurality of tissue grips are resorbable.

60. The method according to claim 52, wherein act (b) includes placing a plurality of first implantable prostheses within the preperitoneal pocket, and act (d) includes positioning the plurality of first implantable prostheses over a plurality of defects in the peritoneum.

61. The method according to claim 52, wherein at least one of act (b) and act (d) occurs prior to at least one of act (c) and act (e).

62. The method according to claim 52, wherein at least one of act (c) and act (e) occurs prior to at least one of act (b) and act (d).

63. The method according to claim 52, wherein act (d) includes positioning one or more first implantable prostheses between the second implantable prosthesis and the peritoneum.

64. The method according to claim 52, wherein the layer of tissue or muscle is the posterior rectus sheath.

65. The method according to claim 52, wherein the method is a robotic transabdominal preperitoneal repair (rTAPP) procedure.

66. The method according to claim 52, wherein the body of the at least one first implantable prosthesis has a maximum width of 5 cm or less.

67. The method according to claim 66, wherein the maximum width of the body ranges from 1 cm to 5 cm.

68. The method according to claim 66, wherein the body has a circular shape with an outer diameter, the maximum width corresponding to the outer diameter.

69. The method according to claim 52, wherein the body is resorbable.

70. The method according to claim 52, wherein the first surface of the body is tissue infiltratable.
